(19) United States
(12) Patent Application Publication
(10) Pub. No.: US 2012/0277402 A1
(43) Pub. Date: Nov. 1, 2012

(54) DINITRO COMPOUND, DIAMINE COMPOUND, POLYAMIDE, AND OPTOELECTRONIC DEVICE

(75) Inventor: Der-Jang Liaw, Taipei City (TW)

(73) Assignee: National Taiwan University of Science and Technology, Taipei (TW)

(21) Appl. No.: 13/196,887

(22) Filed: Aug. 2, 2011

(30) Foreign Application Priority Data
Apr. 26, 2011 (TW) 100114456

(51) Int. Cl. C08G 69/26; C07D 401/10

(52) U.S. Cl. 528/341; 546/276.7

(57) ABSTRACT

A dinitro compound, a diamine compound, a polyamide (PA) derived therefrom, and an optoelectronic device are provided. The PA is fabricated by performing a polycondensation reaction using the foregoing diamine compound and a diacyl chloride compound shown in formula (3) as monomers, and thus represented by formula (4). In formulas (3) and (4), X represents an aromatic group or an aliphatic group. The resultant novel PA can be applied to an optoelectronic device. Since the foregoing compounds have carbazole and pyridine groups, the PA is characterized by excellent solubility, a high glass transition temperature, thermal stability, excellent optical properties, and acidichromism.

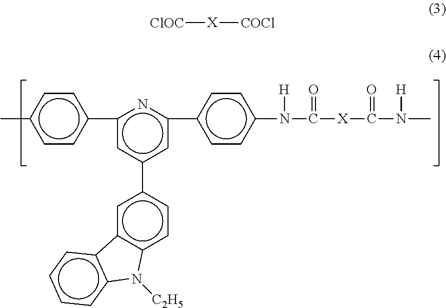

### CROSS-REFERENCE TO RELATED APPLICATION

This application claims the priority benefit of Taiwan application serial no. 100114456, filed on Apr. 26, 2011. The entirety of the above-mentioned patent application is hereby incorporated by reference herein and made a part of this specification.

### BACKGROUND OF THE DISCLOSURE

1. Technical Field

The disclosure is related to a dinitro compound, a diamine compound, a polyamide (PA), and an optoelectronic device, and in particular to a novel dinitro compound which has pyridine heterocyclic and carbazole groups, a novel diamine compound derived from the dinitro compound, a novel PA derived from the diamine compound, and an optoelectronic device which utilizes the PA.

2. Description of Related Art

Since polyamides (PAs) can be applicable to separation membranes for gas-gas separation, liquid-liquid separation, and gas-liquid separation, such type of PA thin films are extremely important in the current age when energy conservation and exploration of new energy are touted. Generally, PAs are able to be co-fabricated with common fibers and inorganic fillings into a reinforced PA composite material. Also, a high-performance and high-strength PA may be fabricated through interactions between PA and metal ions. In order words, by utilizing PA modification technologies, a superiorly heat-resistant, weather-resistant, flame-resistant, and flexible elastic material is able to be fabricated, and a PA with increased dimensional stability can be fabricated as well.

Although heat resistance and mechanical properties of PAs are superb, problems of inferior processability commonly exist. Since PAs have high melting points or softening points, heating and melting cannot be used for processing. In addition, since solubility of PAs is inferior, solubilization by solvents for processing and molding is impractical. Therefore, most aromatic PAs have difficulty in molding and processing.

### SUMMARY

In light of the above, the disclosure provides a dinitro compound, a diamine compound, a novel PA derived therefrom, and an optoelectronic device. Since the foregoing compounds have carbazole and pyridine groups, they are characterized by excellent solubility, high glass transition temperatures, thermal stability, excellent optical properties, and acidichromism.

The disclosure provides a dinitro compound, which is 4-(9-ethyl-3-carbazole)-2,6-bis(4-nitrophenyl)pyridine (CBNPP) and which may be represented by formula (1).

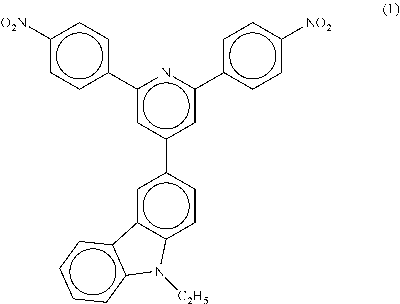

The disclosure provides a diamine compound, which is 4-(9-ethyl-3-carbazole)-2,6-bis(4-aminopheny)pyridine (CBAPP) and which may be represented by formula (2).

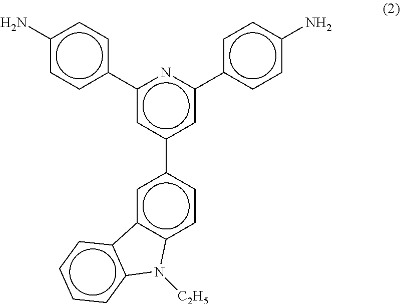

The disclosure provides a PA, which is fabricated by performing a polycondensation reaction using the foregoing diamine compound and a diacyl chloride compound shown in formula (3) as monomers, and thus the PA may be represented by formula (4). In formulas (3) and (4), X represents an aromatic group or an aliphatic group.

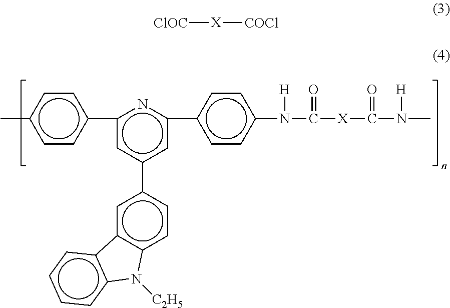

In the PA according to embodiments of the disclosure, in the foregoing formulas (3) and (4), X represents a group selected from formulas (3-1) to (3-11).

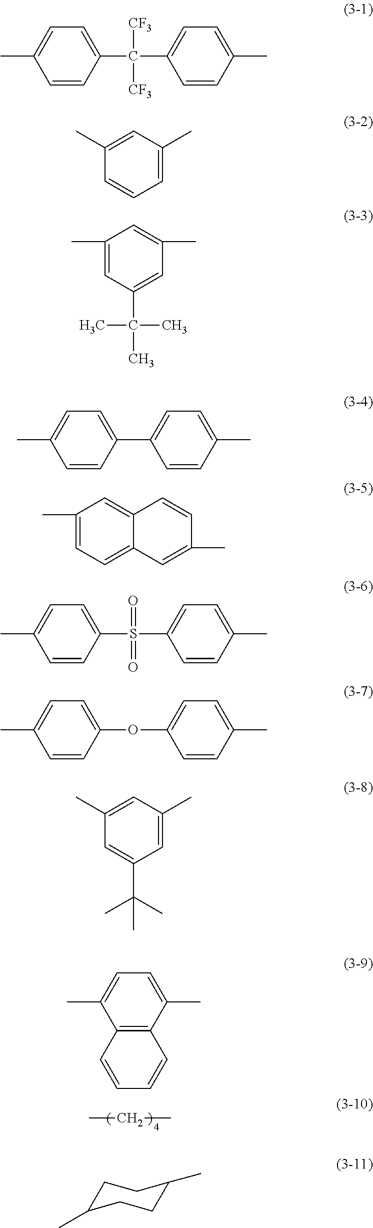

A glass transition temperature of the PA according to embodiments of the disclosure is equal to or greater than 250° C.

The disclosure further provides an optoelectronic device which includes a material layer. The foregoing PA is included in the material layer.

In light of the above description, technical features of the disclosure include the novel diamine compound fabricated with the foregoing dinitro compound, the novel PA fabricated by performing a polycondensation reaction using the foregoing diamine compound and the diacyl chloride compound as monomers, and the optoelectronic device utilizing the PA. Since the novel PA has carbazole and pyridine groups, it is characterized by excellent solubility, a high glass transition temperature, thermal stability, excellent optical properties, and acidichromism, thereby improving processability of the PA and increasing applicability thereof.

In order to make the aforementioned and other objects, features and advantages of the disclosure comprehensible, embodiments accompanied with figures are described in detail below.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 is a schematic ¹H-nuclear magnetic resonance (NMR) spectrum diagram of a dinitro compound (CBNPP) according to an example of the disclosure.

FIG. 2 is a schematic ¹³C-NMR spectrum diagram of a dinitro compound (CBNPP) according to an example of the disclosure.

FIG. 3 is a schematic ¹H-NMR spectrum diagram of a diamine compound (CBAPP) according to an example of the disclosure.

FIG. 4 is a schematic ¹³C-NMR spectrum diagram of a diamine compound (CBAPP) according to an example of the disclosure.

FIG. 5 is a schematic ¹H-NMR spectrum diagram of a PA (PA-10) according to an example of the disclosure.

FIG. 6 is a schematic ¹³C-NMR spectrum diagram of a PA (PA-10) according to an example of the disclosure.

FIG. 7 is a schematic thermogravimetric analysis (TGA) diagram of a PA (PA-10) in nitrogen (N₂) according to an example of the disclosure.

FIG. 8 is a schematic TGA diagram of a PA (PA-10) in air according to an example of the disclosure.

FIG. 9 is a schematic ultraviolet-visible (UV-vis) absorption spectrum diagram of a PA (PA-10) in hydrochloric acid of different concentrations according to an example of the disclosure.

### DETAILED DESCRIPTION OF DISCLOSED EMBODIMENTS

The following describes, according to the disclosure, a novel dinitro compound with heat-resistant heterocyclic rings, a novel diamine derived from the dinitro compound, and PAs fabricated by the foregoing diamine compound and a series of diacyl chloride compounds.

### I. Dinitro Compound

A dinitro compound according to an embodiment of the disclosure is 4-(9-ethyl-3-carbazole)-2,6-bis(4-nitrophenyl)pyridine (CBNPP) and is represented by formula (1).

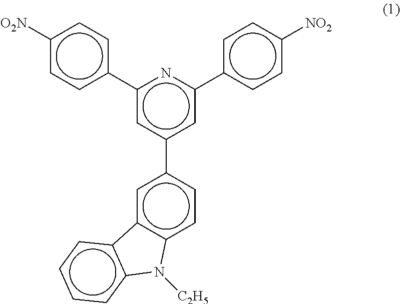

Next, the following description provides an example to describe a method for synthesizing the dinitro compound (CBNPP) and methods for confirming and analyzing a chemical structure of the fabricated compound.

First 75 millimoles (mmol) of 9-ethyl-3-carbazole-carboxaldehyde, 150 mmol of 4′-nitroacetophenone, 1.5 moles (mol) of ammonium acetate, and 360 milliliters (mL) of acetic acid are placed in a flask and react for 1 hour at 90° C., so as to obtain a reaction mixture. Then, heat reflux is performed on the reaction mixture for 72 hours. Next, the reaction mixture is cooled and filtered to obtain a solid portion of the reaction mixture. Afterwards, the obtained solid is recrystallized for 5 times with N,N-dimethyl formamide, so as to obtain a yellow solid material which is the dinitro compound (CBNPP). A measured melting point of the dinitro compound (CBNPP) is 280° C. A yield thereof is about 41%.

A chemical equation for the synthesis of the foregoing dinitro compound (CBNPP) is as follows.

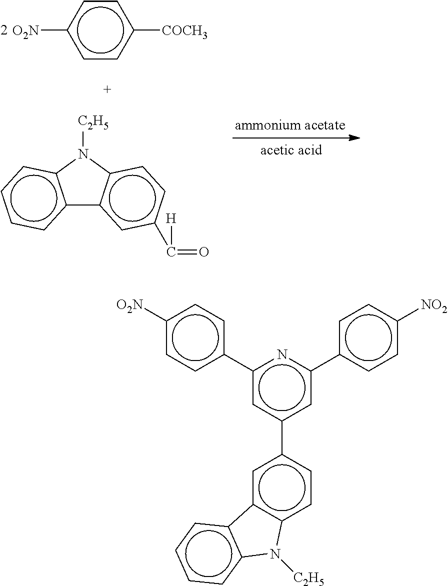

Reaction mechanisms of the foregoing reaction equation are as follows.

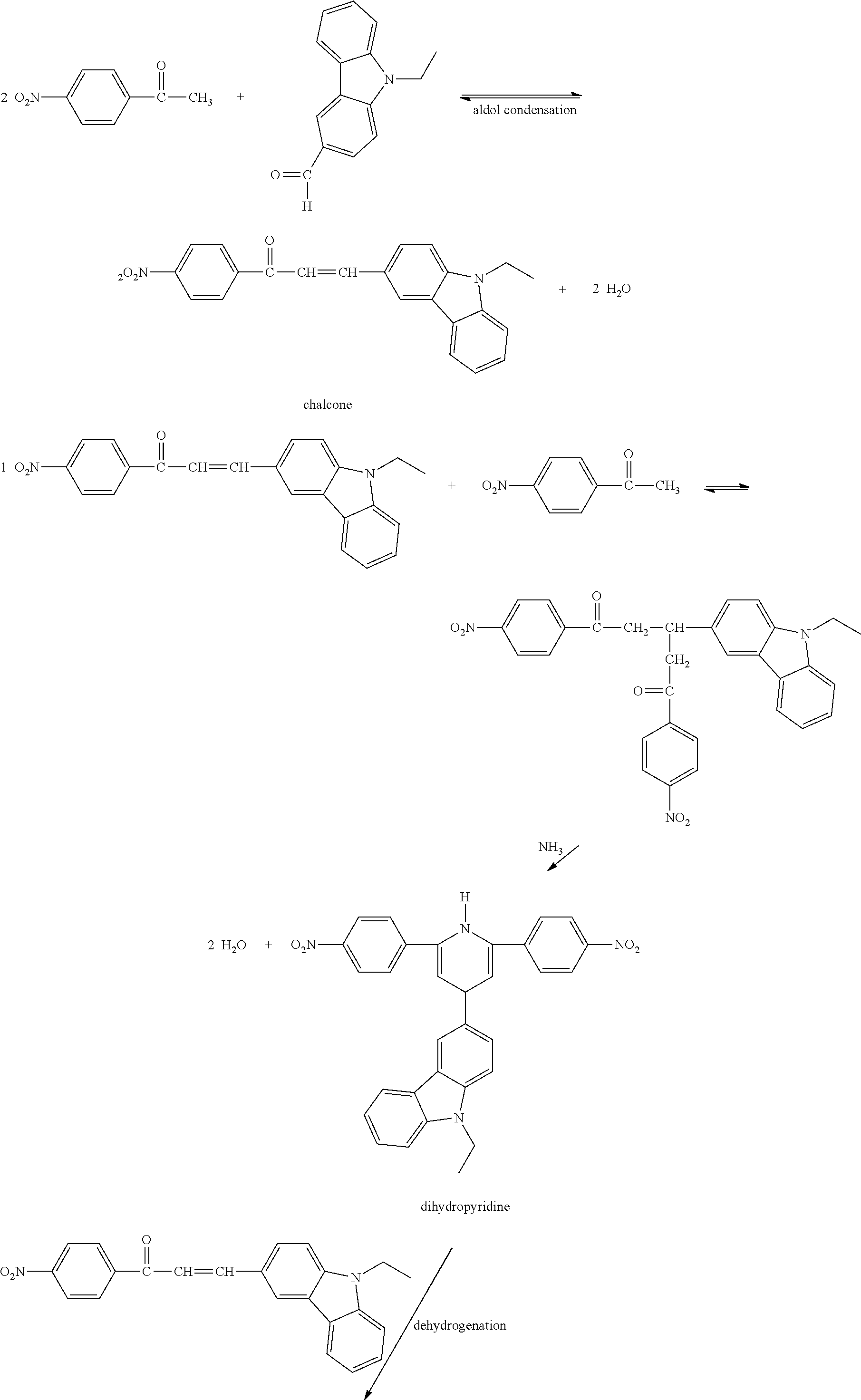

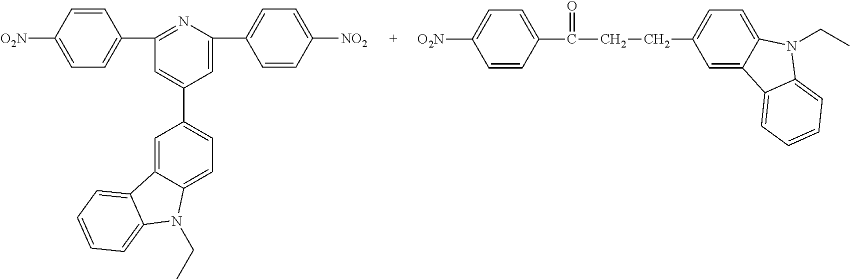

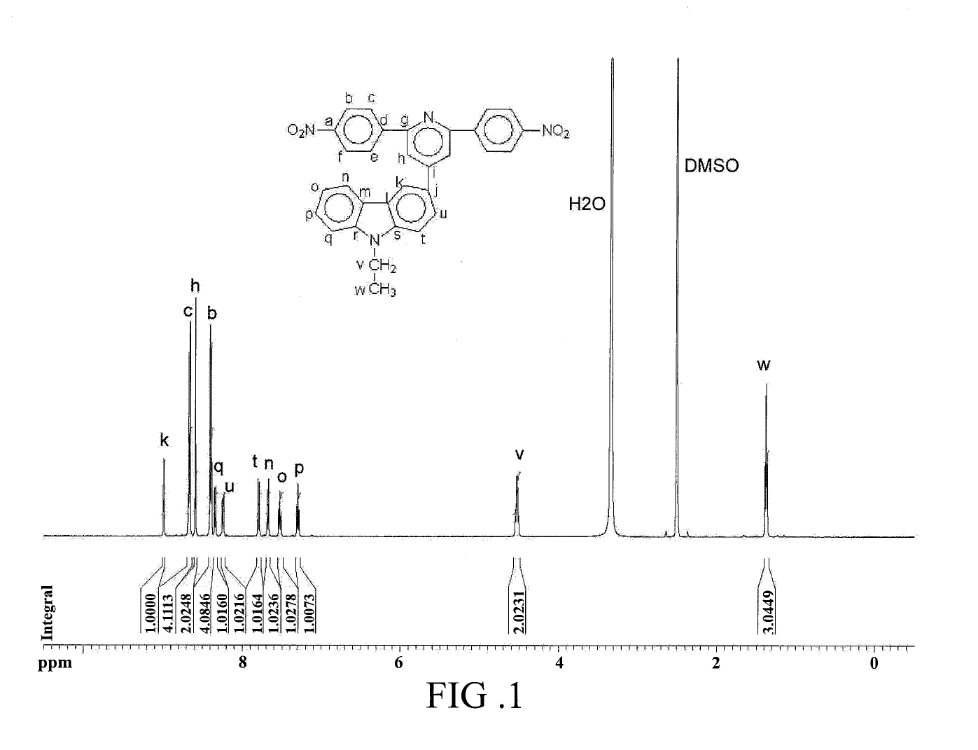

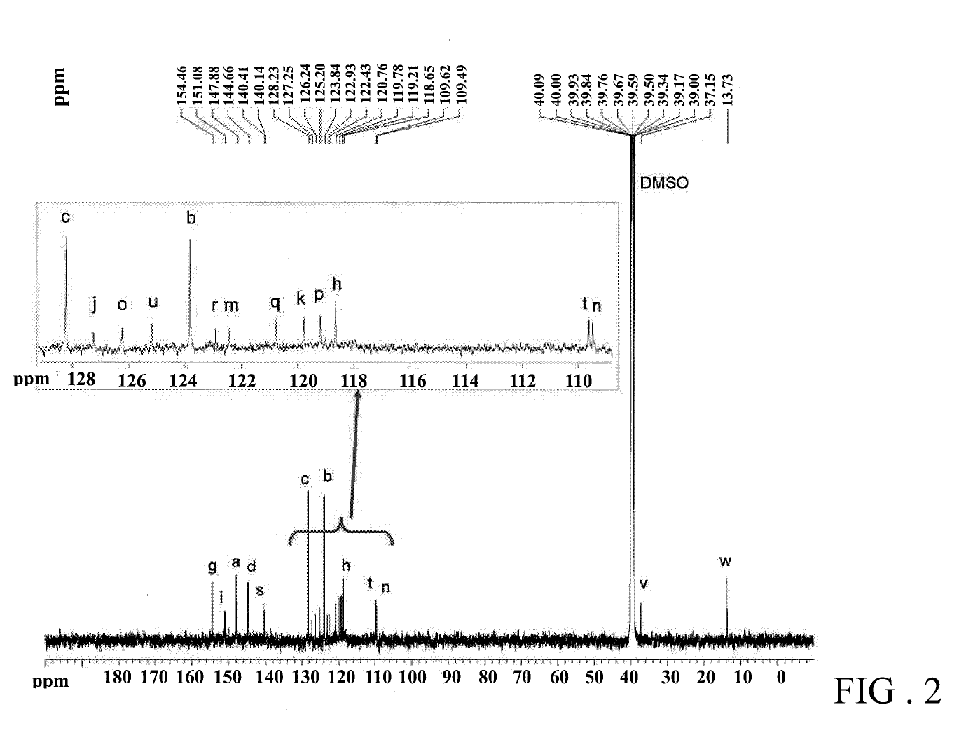

Also, the obtained dinitro compound (CBNPP) is analyzed by ¹H-NMR analysis, ¹³C-NMR analysis of NMR spectrums, and elementary analysis. FIG. 1 is a schematic ¹H-NMR spectrum diagram of the dinitro compound (CBNPP) according to an example of the disclosure. FIG. 2 is a schematic ¹³C-NMR spectrum diagram of the dinitro compound (CBNPP) according to an example of the disclosure. In the NMR spectrums shown in the figures, s denotes a singlet, d denotes a doublet, t denotes a triplet, q denotes a quartet, and m denotes a multiplet.

¹H-NMR (solvent: DMSO-d₆): δ(ppm)=8.99(s, 1H), 8.68-8.66(d, 4H), 8.59(s, 2H), 8.41-8.39(d, 4H), 8.35-8.33(d, 1H), 8.25-8.23(d, 1H), 7.80-7.78(d, 1H), 7.66-7.68(d, 1H), 7.54-7.51(t, 1H), 7.31-7.28(t, 1H), 4.55-4.51(m, 2H), 1.39-1.36(t, 3H).

¹³C-NMR (solvent: DMSO-d₆): δ(ppm)=154.46, 151.08, 147.88, 144.66, 140.41, 140.14, 128.23, 127.03, 126.24, 125.20, 123.84, 122.93, 122.43, 120.76, 119.78, 119.21, 118.65, 109.62, 109.49, 37.15, 13.73.

An elementary analysis result of the dinitro compound (CBNPP) according to the present example is as follows. Theoretical values are C: 72.36%, H: 4.31%, and N: 10.89%; and analytical values are C: 72.29%, H: 4.32%, and N: 10.58%.

### II. Diamine Compound

A diamine compound according to an embodiment of the disclosure is 4-(9-ethyl-3-carbazole)-2,6-bis(4-aminophenyl)pyridine (CBAPP), and is represented by formula (2).

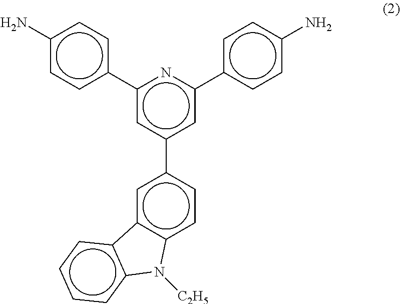

The diamine compound (CBAPP) in formula (2) is fabricated with the dinitro compound (CBNPP) in formula (1) as monomers. It should be noted that the novel diamine compound (CBAPP) has a pyridine heterocyclic group as shown in formula (2-1) and a carbazole group as shown in formula (2-2). Since the pyridine heterocyclic group has heat resistance, and the carbazole group has better optical properties, the diamine compound (CBAPP) has both heat resistance and better optical properties.

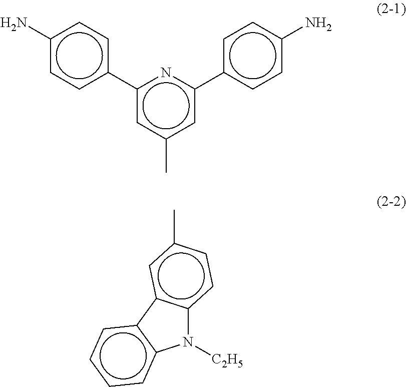

Next, the following description provides an example to describe a method for fabricating the diamine compound (CBAPP) and methods for confirming and analyzing a chemical structure of the fabricated compound.

First, 8.6 mmol of the dinitro compound (CBNPP) monomer, 0.15 gram of 10% activated Pd/C, and 35 mL of ethanol are placed in a flask and mixed.

Then, after the mixture solution is heated to 90° C., 20 mL of hydrazine (H₂NNH₂.H₂O) is slowed added to the solution. After the hydrazine is added, the solution reacts for 24 hours. After the reaction is complete, filtration is performed while the solution is still hot, so as to remove the 10% activated Pd/C and obtain a filtrate. After the obtained filtrate is cooled and precipitates, filtration is performed again to obtain a solid portion. Afterwards, the obtained solid is recrystallized for 2 times by ethanol, so that the white diamine compound (CBAPP) is obtained and then dried in vacuum. A measured melting point of the diamine compound (CBAPP) is 125° C. A yield thereof is about 44%.

A chemical equation for the synthesis of the above diamine compound (CBAPP) is as follows.

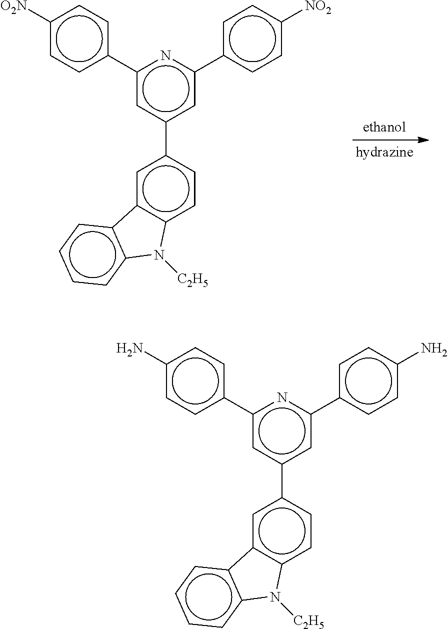

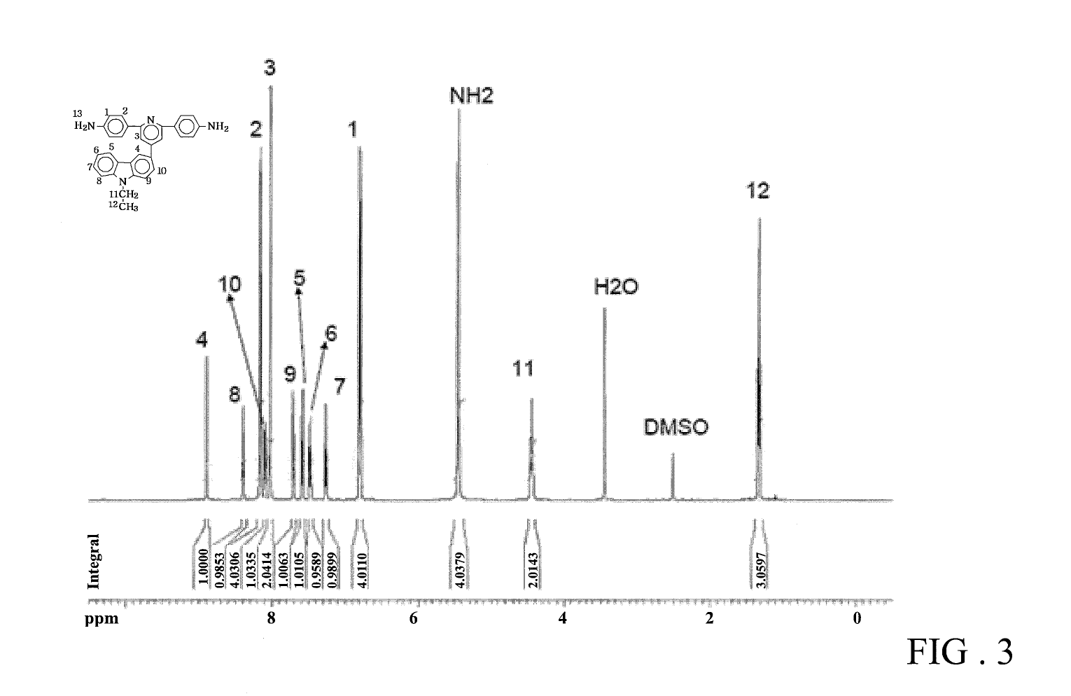

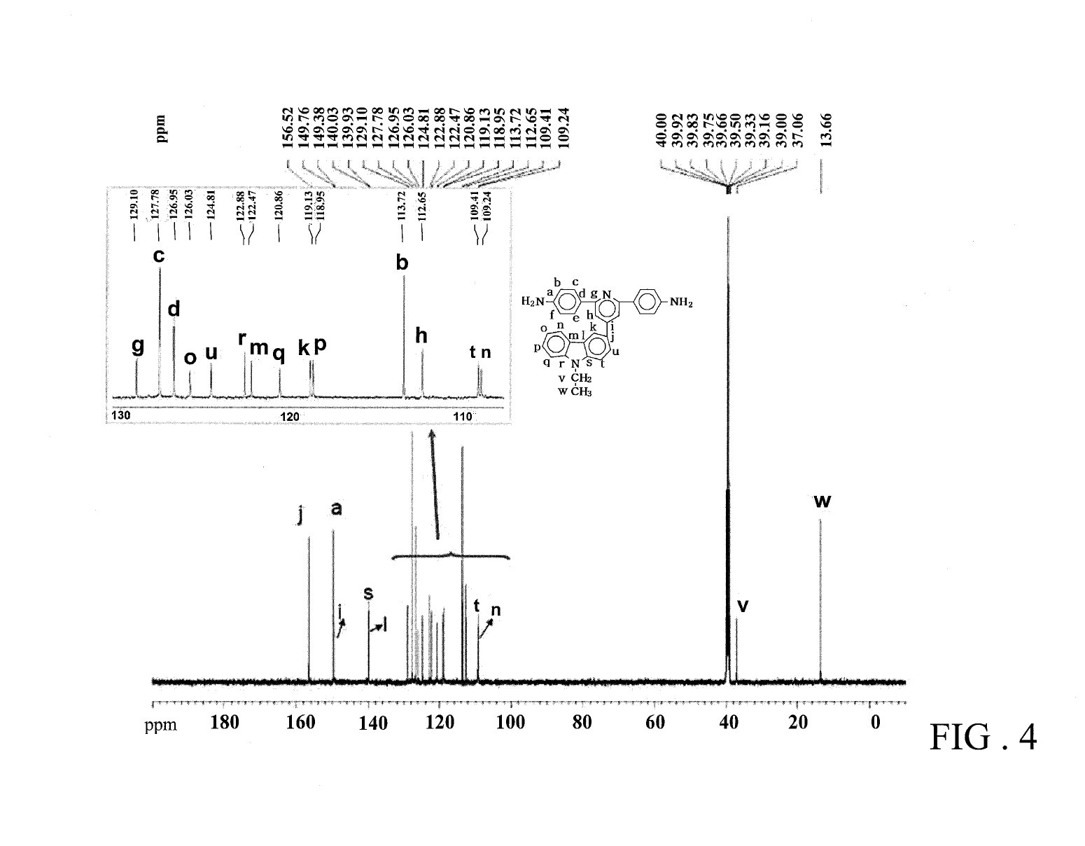

Also, the obtained diamine compound (CBAPP) is analyzed by ¹H-NMR analysis, ¹³C-NMR analysis of NMR spectrums, and elementary analysis. FIG. 3 is a schematic ¹H-NMR spectrum diagram of the diamine compound (CBAPP) according to an example of the disclosure. FIG. 4 is a schematic ¹³C-NMR spectrum diagram of the diamine compound (CBAPP) according to an example of the disclosure.

¹H-NMR (solvent: DMSO-d₆): δ (ppm)=8.89 (s, 1H), 8.40-8.38 (d, 1H), 8.17-8.38 (d, 4H), 8.10-8.08 (d, 1H), 8.03 (s, 2H), 7.71-7.70 (d, 1H), 7.60-7.58 (d, 1H), 7.49-7.46 (t, 1H), 7.28-7.25 (t, 1H), 6.81-6.79 (d, 4H), 5.44 (s, 4H), 4.46-4.42 (m, 2H), 1.33-1.31 (t, 3H).

¹³C-NMR (solvent: DMSO-d₆): δ(ppm)=156.52, 149.76, 149.38, 140.93, 139.93, 129.10, 127.78, 126.95, 126.03, 124.81, 122.88, 122.47, 120.86, 119.13, 118.95, 113.72, 112.65, 109.41, 109.24, 37.06, 13.66.

An elementary analysis result of the diamine compound (CBAPP) according to the present example is as follows. Theoretical values are C: 81.91%, H: 5.77%, and N: 12.33%; and analytical values are C: 81.84%, H: 5.86%, and N: 12.07%.

### III. PA

A PA according to an embodiment of the disclosure is fabricated by performing a polycondensation reaction using the foregoing diamine compound (CBAPP) shown in formula (2) and a diacyl chloride compound shown in formula (3) as monomers, and thus represented by formula (4).

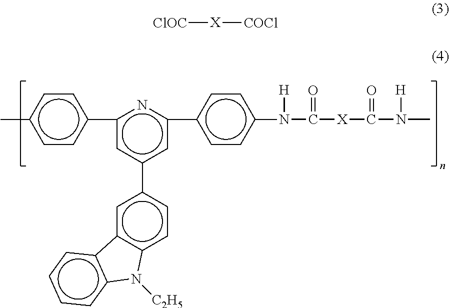

In formulas (3) and (4), X represents an aromatic group or an aliphatic group. According to an embodiment, in formulas (3) and (4), X may represent a group shown in formula (3-1), formula (3-2), formula (3-3), formula (3-4), formula (3-5), formula (3-6), formula (3-7), formula (3-8), formula (3-9), formula (3-10), or formula (3-11) as listed below. It should be noted that since the novel PA is derived from the diamine compound (CBAPP) shown in formula (2), the novel PA is also characterized by the heat resistance and superb optical properties of the diamine compound (CBAPP).

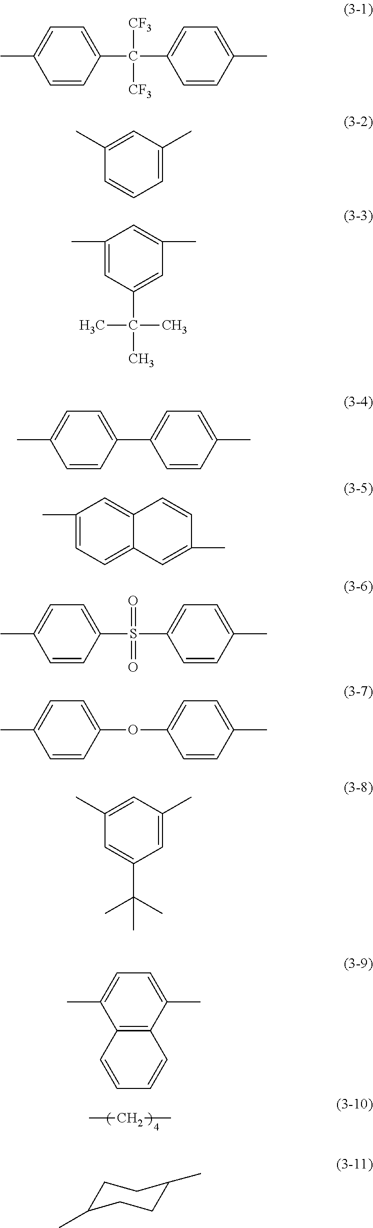

Next, examples for fabricating the PA according to the disclosure are illustrated. The following is a more detailed description according to examples, but the disclosure is not limited to these examples.

### EXAMPLE 1

### Synthesis of PA (PA-1)

0.760 mmol (0.345 gram) of the diamine compound (CBAPP) is dissolved in 3.8 mL of N-methyl-2-pyrrolidinone (NMP) solvent, and 0 8 mL of propylene oxide is added. Next, 0.760 mmol (0.326 gram) of 2,2′-bis(4-carboxyphenyl)hexa-fluoropropane acid chloride monomer is slowly added to the solution. In other words, the structure in formula (3-1) is chosen as X in the diacyl chloride compound in formula (3), so as to synthesize the PA (PA-1). After the foregoing mixture solution reacts at room temperature for 6 hours, the mixture solution is poured into large quantities of methanol for precipitation. The polymer is then washed by methanol and dried at 100° C. in vacuum, thereby obtaining the PA (PA-1).

The obtained PA (PA-1) is analyzed by ¹H-NMR analysis, ¹³C-NMR analysis of NMR spectrums, and elementary analysis.

¹H-NMR (500 MHz, solvent: DMSO-d₆): δ(ppm)=10.1 (amide NH), 8.92 (s, 1H, H_{d}), 8.35-8.37 (d, 5H, H_{g}+H_{b}), 8.25 (s, 1H, H_{c}), 8.14-8.16 (s, 1H, Hₑ), 7.83-7.85 (d, 2H, Hₐ), 7.74-7.75 (d, 1H, H_{f}), 7.61-7.62 (d, 1H, Hᵢ), 7.48 (s, 1H, Hⱼ), 7.25 (s, 1H, Hₕ), 4.49 (s, 2H, Hₖ), 2.45 (s, 2H, Hₘ), 1.71-1.76 (m, 2H, Hₙ), 1.34 (s, 3H, Hₗ).

¹³C-NMR (500 MHz, solvent: DMSO-d₆): δ(ppm)=171.3, 155.8, 150.0, 140.3, 140.2, 140.1, 133.96, 128.8, 128.3, 128.1, 127.3, 126.0, 124.9, 122.9, 122.5, 120.8, 119.4, 118.9, 115.0, 109.5, 109.3, 37.1, 36.4, 25.1, 13.7.

An elementary analysis result of the PA (PA-1) according to the present example is as follows. Theoretical values (C₃₁H₂₆N₄) are C: 71.0%, H: 4.1%, and N: 6.9%; and analytical values are C: 69.3%, H: 3.8%, and N: 7.1%.

Moreover, identification and property analysis of a chemical structure of the PA (PA-1) are as follows.

Thermal property: a glass transition temperature is about 200° C. ; in nitrogen, a temperature at which 10% degradation occurs is about 430° C.; in air, a temperature at which 10% degradation occurs is about 435° C.

Viscosity: in N,N-dimethylacetamide (DMAc), an intrinsic viscosity is 0.76 dL⁻¹ g (solution concentration: 0.5 g dL⁻¹; measurement temperature: 30° C.).

Solubility: the PA (PA-1) is soluble in solvents such as N-methyl-2-pyrollidone (NMP), N,N-dimethylacetamide (DMAc), N,N-dimethylformamide (DMF), dimethyl sulfoxide (DMSO), tetrahydrofuran (THF), and m-cresol.

Thin film mechanical property: a tensile strength is 40 MPa, an elongation is 2.9%, and a tensile coefficient is 1.7 GPa.

### EXAMPLE 2

### Synthesis of PA (PA-10)

0.883 mmol of the diamine compound (CBAPP) is dissolved in 4.0 mL of NMP solvent, and 0.4 mL of propylene oxide is added. Next, 0.883 mmol of cyclobutanedioyl chloride monomer is slowly added to the solution. In other words, the structure in formula (3-10) is chosen as X in the diacyl chloride compound in formula (3), so as to synthesize the PA (PA-10). Next, after the foregoing mixture solution reacts at room temperature for 6 hours, the mixture solution is poured into large quantities of methanol for precipitation. The polymer is then washed by methanol and dried at 100° C. in vacuum, thereby obtaining the PA (PA-10).

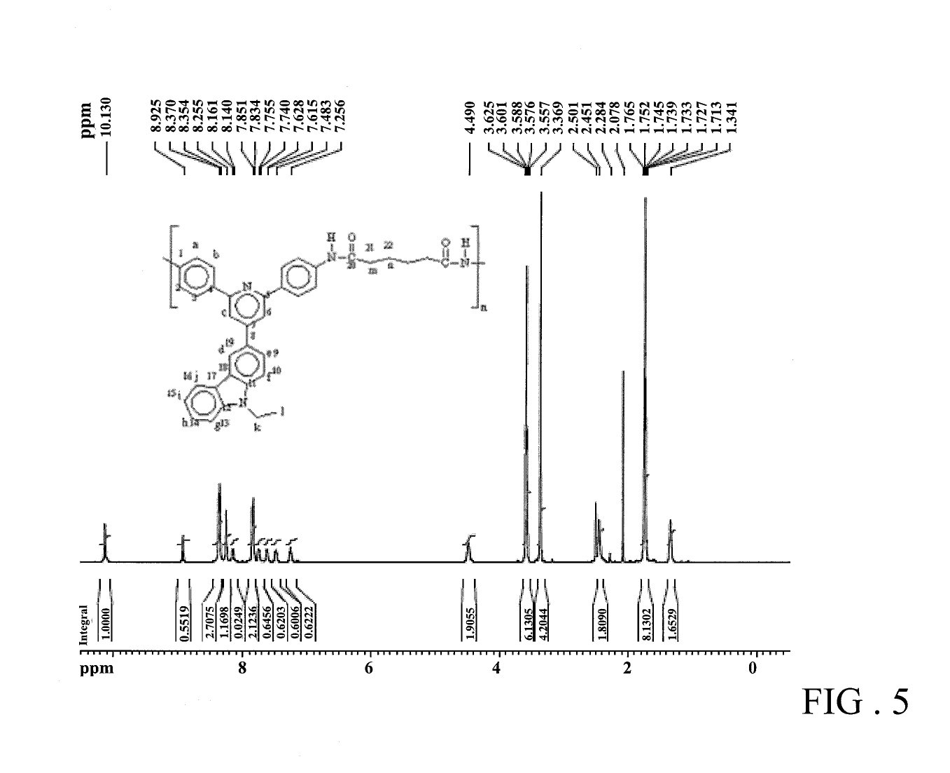

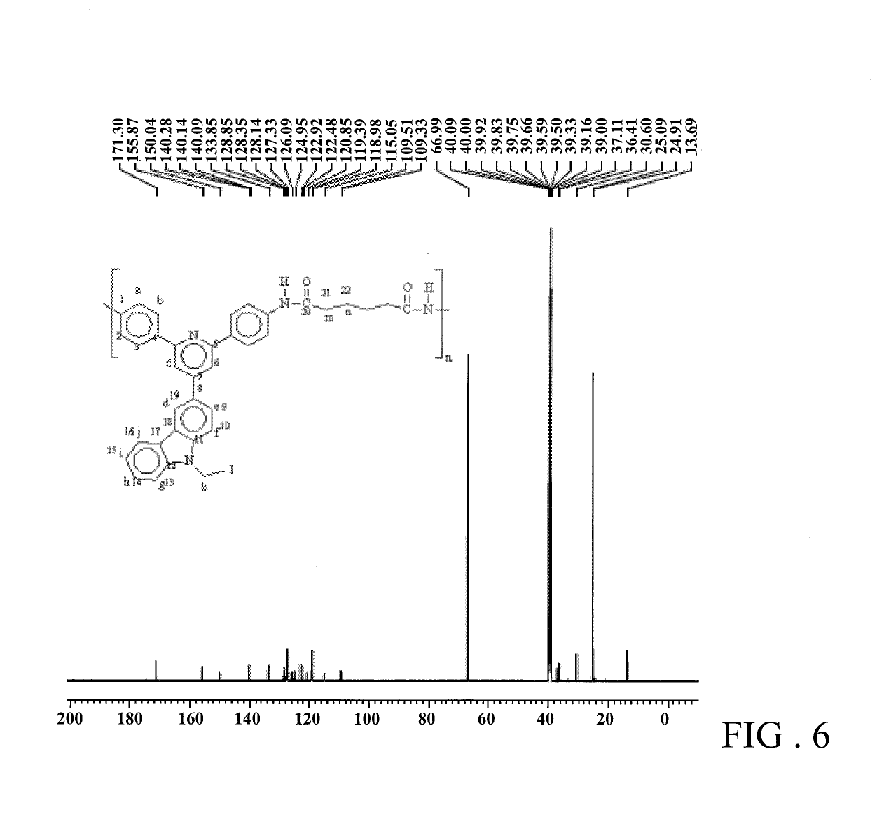

The obtained PA (PA-10) is analyzed by ¹H-NMR analysis and ¹³C-NMR analysis of NMR spectrums. FIG. 5 is a schematic ¹H-NMR spectrum diagram of the PA (PA-10) according to an example of the disclosure. FIG. 6 is a schematic ¹³C-NMR spectrum diagram of the PA (PA-10) according to an example of the disclosure.

¹H-NMR (solvent: DMSO-d₆): δ(ppm)=1.34, 1.71, 2.45, 4.50, 7.25, 7.48, 7.61, 7.62, 7.74, 7.75, 7.83, 7.85, 8.14, 8.16, 8.25, 8.35, 8.37, 8.92.

¹³C-NMR (solvent: DMSO-d₆): δ(ppm)=171.3, 155.8, 150.1, 140.3, 140.1, 133.6, 128.8, 128.3, 128.1, 127.3, 126.1, 125.0, 123.0, 122.5, 120.8, 119.4, 118.9, 115.0, 109.5, 109.3, 39.3, 36.4, 25.1, 13.7.

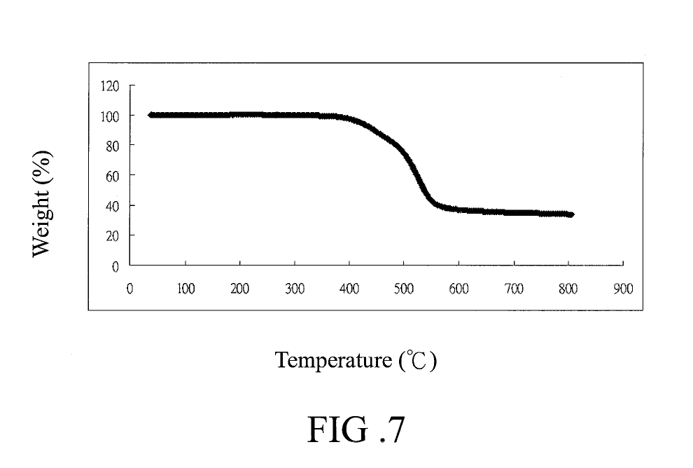

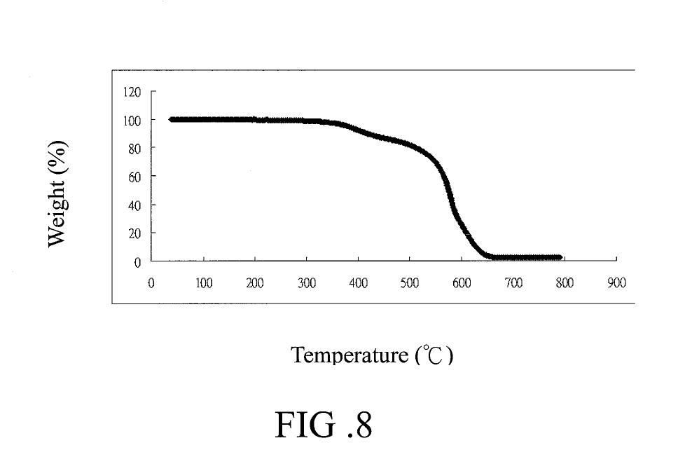

Moreover, identification and property analysis of a chemical structure of the PA (PA-10) are as follows. FIG. 7 is a schematic TGA diagram of the PA (PA-10) in nitrogen according to an example of the disclosure. FIG. 8 is a schematic TGA diagram of the PA (PA-10) in air according to an example of the disclosure.

Thermal property: a glass transition temperature is about 250° C.; in nitrogen, a temperature at which 10% degradation occurs is about 450° C.; in air, a temperature at which 10% degradation occurs is about 420° C.

Viscosity: in DMAc, an intrinsic viscosity is 0.44 dL⁻¹ g (solution concentration: 0.5 g dL⁻¹; measurement temperature: 30° C.).

Solubility: the PA (PA-10) is soluble in solvents such as NMP, pyridine, DMAc, DMF, DMSO, and m-cresol.

Thin film mechanical property: a tensile strength is 40 MPa, an elongation is 3%, and a tensile coefficient is 2.1 GPa.

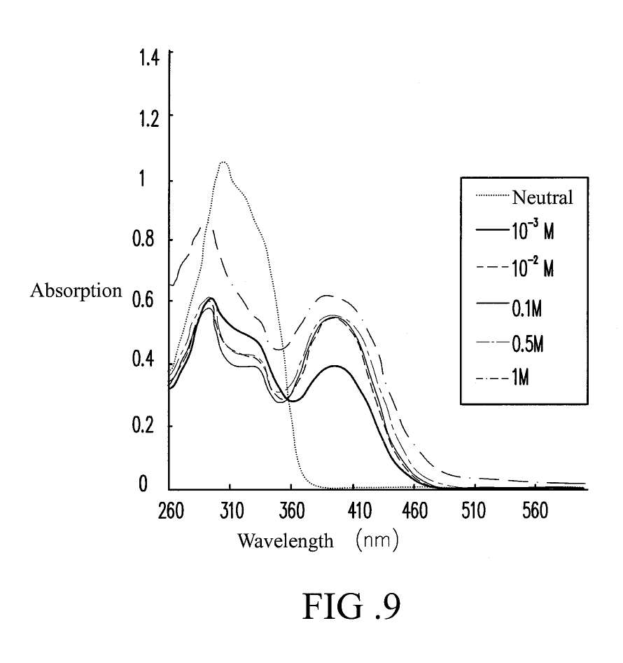

FIG. 9 is a schematic UV-vis absorption spectrum diagram of the PA (PA-10) in hydrochloric acid of different concentrations according to an example of the disclosure. According to FIG. 9, when dripping hydrochloric acid of different concentrations into the PA (PA-10) in the THF solution, absorption at the wavelength of 310 nm in the UV-vis absorption spectrum diagram gradually decreases, and new peaks occur at wavelengths of 295 nm and 395 nm. In other words, an acid may be used to protonate the PA (PA-10), so as to change a fluorescent wavelength of the PA (PA-10). Fluorescence generated by the PA (PA-10) when irradiated by UV light changes from blue to yellow, so that the PA (PA-10) is characterized by acidichromism.

Furthermore, the foregoing PA can be applied to an optoelectronic device. The optoelectronic device according to an embodiment of the disclosure may include a material layer, wherein the material layer contains the PA with superb properties illustrated above.

It should be noted that the PA according to the foregoing embodiments is fabricated by performing a polycondensation reaction using the diamine compound and the diacyl chloride compound as monomers. Since the foregoing PA has the pyridine heterocyclic group and the carbazole group, the PA is characterized by heat resistance and superb optical properties and also by properties such as excellent solubility, a high glass transition temperature, thermal stability, optoelectrical properties, and acidichromism. In further detail, compared with the benzene ring, pyridine is a substituent of aromatic heterocycle and has lone pair electrons in sp2 orbital of the nitrogen atom, so that a polymer material which has a pyridine heterocyclic group has increased electron affinity and improved electron transport. Therefore, in the PA according to the disclosure, after the pyridine heterocyclic group, the carbazole group and derivatives thereof are introduced into the polymer segment, owing to protonation of the lone pair electrons, an optoelectronic material is obtained with superb properties and characterized by excellent solubility, a high glass transition temperature, thermal stability, and excellent optical properties.

In light of the above, the dinitro compound, the diamine compound, the PA derived therefrom, and the optoelectronic device have at least one of the following advantages:

The dinitro compound, the diamine compound, and the PA derived therefrom according to an embodiment of the disclosure have pyridine heterocyclic groups, so that they are characterized by heat resistance.

The dinitro compound, the diamine compound, and the PA derived therefrom according to an embodiment of the disclosure have carbazole groups, so that they are characterized by excellent optical properties.

The PA according to an embodiment of the disclosure is characterized by excellent solubility, a high glass transition temperature, thermal stability, excellent optical properties, and acidichromism, thereby improving processability of the PA and increasing applicability thereof.

It will be apparent to those skilled in the art that various modifications and variations can be made to the disclosure without departing from the scope or spirit of the disclosure. In view of the foregoing, it is intended that the disclosure cover modifications and variations of this disclosure provided they fall within the scope of the following claims and their equivalents.

1. A dinitro compound, which is 4-(9-ethyl-3-carbazole)-2,6-bis(4-nitrophenyl)pyridine (CBNPP) and represented by formula (1).

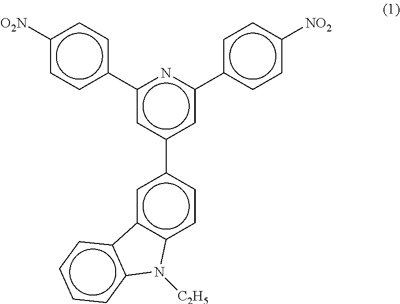

2. A diamine compound, which is 4-(9-ethyl-3-carbazole)-2,6-bis(4-aminophenyl)pyridine (CBAPP) and represented by formula (2).

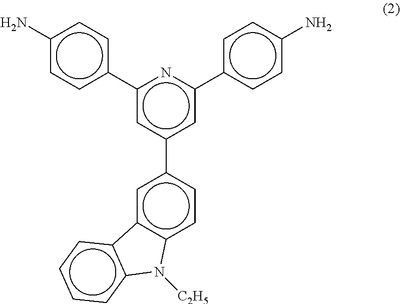

3. A polyamide (PA), which is fabricated by performing a polycondensation reaction using a diamine compound shown in formula (2) and a diacyl chloride compound shown in formula (3) as monomers, wherein the PA is represented by formula (4), 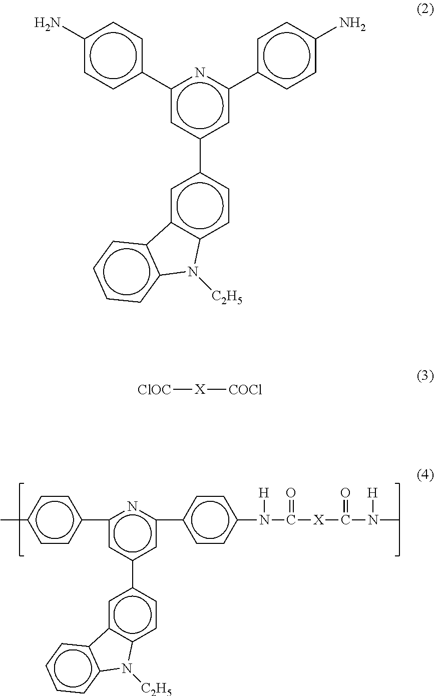
wherein in formulas (3) and (4), X represents an aromatic group or an aliphatic group.

4. The PA as claimed in claim 3, wherein in formulas (3) and (4), X represents a group selected from formulas (3-1) to (3-11):

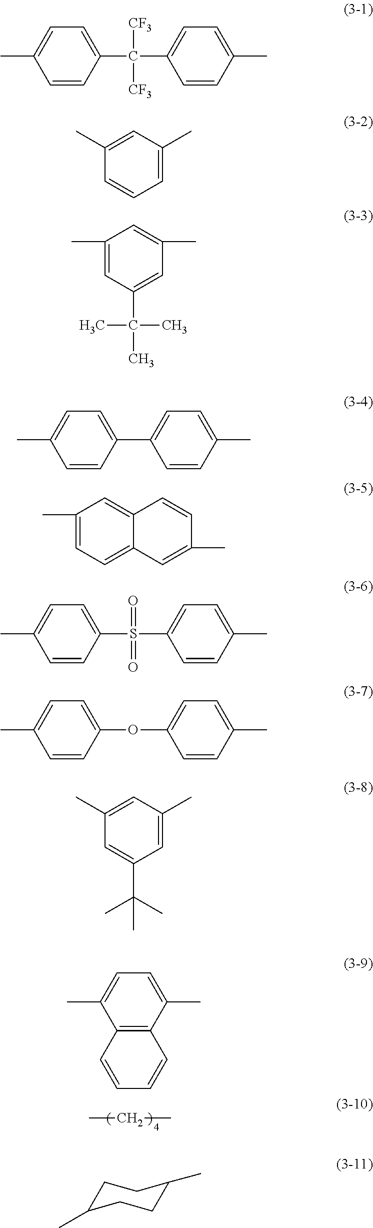

5. The PA as claimed in claim 3, wherein a glass transition temperature of the PA is equal to or greater than 250° C.

6. An optoelectronic device, comprising a material layer, wherein the material layer contains the PA as claimed in claim 3.
